(19) United States
(12) Patent Application Publication
(10) Pub. No.: US 2025/0336544 A1
(43) Pub. Date: Oct. 30, 2025

(54) Determination of Occult Sepsis

(71) Applicant: Beckman Coulter, Inc., Brea, CA (US)

(72) Inventors: Scott Levin, Washington, DC (US); Maxim Shatsky, Castro Valley, CA (US); Anthony Deangelo, Lutherville, MD (US); HuaHua Zheng, Boston, MA (US)

(21) Appl. No.: 19/195,065

(22) Filed: Apr. 30, 2025

Related U.S. Application Data
(60) Provisional application No. 63/640,759, filed on Apr. 30, 2024.

(51) Int. Cl. G16H 50/30; A61B 5/00; G16H 50/20; G16H 80/00

(52) U.S. Cl. CPC G16H 50/30; A61B 5/412; G16H 50/20; G16H 80/00

(57) ABSTRACT

A computer-implemented method of determining, for a subject, a risk of having an occult sepsis is described. The method comprises obtaining at least one sepsis score indicative of a risk of the subject developing a sepsis event; confirming that the obtained at least one sepsis score meets at least one threshold value for the at least one sepsis score; and analysing a set of subject data indicative of a health state of the subject with respect to one or more occult sepsis criteria indicative of a risk of the subject having an occult sepsis.

### TECHNICAL FIELD

The present disclosure generally relates to the field of clinical decision support. In particular, the present disclosure relates to a computer-implemented method of determining, for a subject, a risk of having occult sepsis. The present disclosure further relates to a computing device or system configured to carry out steps of such method, to a corresponding computer program causing the computing device or system to perform steps of such method, and to a computer-readable medium storing such computer program.

### BACKGROUND OF THE INVENTION

Sepsis refers to a serious medical condition that occurs when the response of a subject's body causes inflammation throughout the body. Sepsis is triggered by an infection, which can be bacterial, viral, fungal, or caused by other pathogens. The body's immune system responds to the infection, but if the response is out of balance, it can cause widespread inflammation, leading to blood clotting, leaky blood vessels, and impaired blood flow, which can deprive organs of nutrients and oxygen. Therefore, sepsis can lead to shock, organ failure, and even death if not recognized and treated promptly and effectively.

The key to survival and the avoidance of serious complications is generally the early diagnosis and treatment of sepsis. However, this can be difficult and challenging even for experienced clinicians to achieve because initial symptoms are often subtle and non-specific. Therefore, sepsis detection and antibiotic treatment can in practice often be delayed up to by several hours and may thus contribute to overall mortality recorded at clinics and hospitals.

Additional challenges arise for the so-called occult sepsis, which is a special form or type of sepsis that refers to a condition where a subject actually has a sepsis infection or is developing a sepsis, but the usual signs and symptoms of sepsis are not overtly apparent, are minimally pronounced or even not recognizable at the subject. The term “occult” in this context means hidden or not easily detected. Thus, occult sepsis may not only be challenging to detect, but can be particularly dangerous because the absence of clear symptoms can lead to a delay in diagnosis and treatment, which can increase the risk of severe complications.

Although various clinical risk scores like the Systemic Inflammatory Response Syndrome (SIRS), the quick Sequential Organ Failure Assessment (qSOFA) and the Modified Early Warning Assessment (MEWS) scores are often employed for sepsis screening, these simple rule-based scores typically have poor overall performance for early sepsis detection, and particularly do not provide an indication about occult sepsis.

### SUMMARY OF THE INVENTION

Therefore, it may be desirable to provide for a computer-implemented method and computing device facilitating reliable detection of occult sepsis, for example thereby facilitating early detection, and hence early intervention and treatment of subjects.

This is achieved by the subject matter of the independent claims, wherein further exemplary embodiments are included in the dependent claims and the following description.

A first aspect of the present disclosure relates to a computer-implemented method of determining a subject's risk of having occult sepsis. A second aspect of the present disclosure relates to a computing device or system configured to carry out steps of the method according to the first aspect. A third aspect of the present disclosure relates to a computer program comprising instructions, which, when the program is executed by a computing device, instruct the computing device to perform steps of the method according to the first aspect. A fourth aspect of the present disclosure relates to a computer-readable medium, e.g. a non-transitory computer-readable medium, storing such computer program. It is emphasized that any disclosure presented herein with reference to one aspect of the present disclosure equally applies to any other aspect of the present disclosure, unless explicitly stated otherwise. In particular, any feature, function, step and advantage described herein with respect to the method according to the first aspect, can be a feature, function, element and/or advantage of the computing device according to the second aspect of the present disclosure, and vice versa. Accordingly, any step of the method according to the first aspect can be implemented as functional feature or functionality of the computing device according to the second aspect of the present disclosure, and vice versa. Further, any feature and/or step described herein with reference to the method according to the first aspect can be performed or executed by the computing device according to the second aspect.

According to the first aspect of the present disclosure, there is provided a computer-implemented method of determining, detecting and/or assessing, for a subject, a risk of having an occult sepsis. The method comprises:
obtaining at least one sepsis score indicative of a risk of the subject developing a sepsis and/or a sepsis event;
confirming that the obtained at least one sepsis score meets at least one threshold value for the sepsis score; and
analysing and/or evaluating a set of subject data indicative of a health state of the subject with respect to one or more occult sepsis criteria, the one or more sepsis criteria being indicative of a risk of the subject having an occult sepsis.

In particular, the at least one sepsis score may be obtained and/or received, for example by or at the computing device. It may be determined whether the obtained at least one sepsis score meets the at least one threshold value for the at least one sepsis score, for example reaches, exceeds or falls below the at least one threshold value for the at least one sepsis score. Further, the method may comprise analysing and/or evaluating, upon confirming that the at least one sepsis score meets the at least one threshold value, the set of subject data with respect to and/or in terms of the one or more occult sepsis criteria that are indicative of the risk of the subject having an occult sepsis.

In particular, occult sepsis is a special form or type of sepsis that refers to a condition where a subject actually has a sepsis infection or is developing a sepsis, but the usual signs and symptoms of sepsis are not overtly apparent or are minimally pronounced at the subject. Therefore, occult sepsis is hardly recognizable or detectable by Health Care Providers (HCPs), such as clinicians or medical doctors, e.g. at clinics, hospitals or emergency departments (EDs).

The method and device described herein can provide for a systematic computer-implemented approach and tool for assessing the risk for occult sepsis for a given subject. In turn, the method and device described herein can allow for an accurate and reliable detection of occult sepsis, in particular an early detection of occult sepsis at the subject at a time when only minor or even no symptoms related to sepsis may be recognizable at the subject. The present disclosure, therefore, can provide for an improved clinical decision support system, for example allowing to efficiently, reliably and/or accurately stratify a subject in terms of the risk of having occult sepsis. In turn, the method and device described herein can facilitate early intervention and treatment of subjects when this can be most beneficial and lead to the best clinical outcomes.

In the context of the present disclosure, the term “subject” is to be construed broadly and may generally refer to a vertebrate. Hence, the present disclosure is not limited to specific types of subjects, such as human subjects, but rather includes human and animal subjects. While not being limited to human subjects, the term “subject” may be synonymously or interchangeably used herein with the term “individual” or “patient”. Further, it is noted that a subject, patient or individual in the context of the present disclosure can include subjects, patients or individuals of any age, race, or demographic.

As used herein, a “sepsis event” may refer to the onset of sepsis, for example as indicated by one or more conditions or symptoms of a subject. While the term “sepsis” may generally refer to sepsis as a disease, it is noted that at least in some instances the terms “sepsis” and “sepsis event” can be interchangeably used herein.

The sepsis score may be indicative, descriptive or reflective of the risk, likelihood and/or probability for sepsis or a sepsis event to occur at the subject. Alternatively or additionally, the sepsis score may be indicative, descriptive or reflective of the risk, likelihood and/or probability for the subject having sepsis and/or of developing a sepsis event.

It is noted that the method of the first aspect can involve obtaining a plurality of sepsis scores. Thus, any reference to “a”, “an” or “the” sepsis score herein includes a plurality of sepsis scores. The same applies to the at least one threshold value for the at least one sepsis score.

In an example, the sepsis score may refer to a numerical measure and/or continuous variable indicative of the risk, likelihood and/or probability for sepsis or a sepsis event to occur at the subject, for example within a predetermined period of time, e.g. from a time of assessing the risk for occult sepsis and/or from a time of computing or determining the sepsis score. The predetermined period of time may be between 0 hours and 48 hours, for example between 0 hours and 24 hours. Preferably the predetermined period of time may be between 0 hours and 12 hours or between 12 hours and 24 hours. It should be noted that “0 hours” may refer to or denote the time point of assessing occult sepsis, respectively, the time point of carrying out the method of determining occult sepsis disclosed herein, and/or the time point of computing or determining the sepsis score. Alternatively or additionally, “0 hours” may refer to the time point of determination of the at least one sepsis score.

In a non-limiting example, the sepsis score may be provided on an arbitrary scale ranging from a minimum value, for example zero or 0, to a maximum value, for example one, 10 or 100. Any other scale, including relative and absolute scales, can be used instead to represent the sepsis score. Accordingly, the sepsis score may be provided as a continuous variable.

Alternatively or additionally, the sepsis score may be indicative of and/or associated with a risk level or risk tier, such as low sepsis risk, medium sepsis risk, and a high sepsis risk. It should be noted that the present disclosure is not limited to three levels or tiers of the sepsis risk, but any plurality of levels or tiers, i.e. two or more levels or tiers, can be used instead.

Further, as used herein, a threshold value for the sepsis score can generally refer to a particular prerequisite and/or requirement, for example a predefined prerequisite and/or requirement, which the sepsis score can fulfil or meet. For instance, a threshold value for the sepsis score can relate to a particular numerical value, for example a predefined numerical value, of the sepsis score. Such numerical value can be given as absolute value or relative value, for example normalized to a maximum value of the sepsis score. Alternatively or additionally, the threshold value for the sepsis score can relate to one or more particular, for example predefined, risk levels or tiers of the sepsis score, such as a low, medium or high sepsis risk.

In an example, the step of confirming that the obtained sepsis score meets the at least one threshold value for the sepsis score can include evaluating and/or analysing the sepsis score with respect to the at least one threshold value. This can, for example, include comparing the numerical value of a sepsis score to a numerical value of the threshold value. Depending on the scale of the sepsis score chosen, it may be determined whether the sepsis score reaches, exceeds or falls below a particular threshold value for the sepsis score. For instance, when the sepsis score is given on a positive scale, it may be determined whether the sepsis score reaches and/or exceeds the threshold value. On the other hand, when the sepsis score is given on a negative scale, it may be determined whether the sepsis score reaches and/or falls below the threshold value. Alternatively or additionally, evaluating and/or analysing the sepsis score with respect to the at least one threshold value can include determining and/or assessing whether a particular risk level or tier, as indicated by or associated with the sepsis score, corresponds to or matches a particular level or tier of sepsis risk, as indicated by or associated with the threshold value for the sepsis score. For instance, the threshold value may refer or indicate a medium sepsis risk, so that a sepsis score that meets the threshold value refers to or indicates a medium or high sepsis risk. For example, the threshold value may refer or indicate a high sepsis risk so that a sepsis score meeting the threshold value refers to or indicates a high sepsis risk.

It should be noted that a plurality of threshold values for the sepsis score may be used, such as a lower threshold and an upper threshold, which may for example indicate a certain range of values for the sepsis score or a certain set of a plurality of tiers or levels for the sepsis risk.

In the context of the present disclosure the “set of subject data” (synonymously or interchangeably used herein with “subject data”) may refer to a set of data or dataset including one or more health-related parameters (also referred to herein as “subject parameters”) indicative of, descriptive of or related to the health state of the subject. The expressions “health-related parameters” and “subject parameters” are to be construed broadly and do not only encompass parameters directly related to the health state, but can include any parameter characteristic or information descriptive of or associated with the subject, such as e.g. age, gender, or demographic of the subject. For instance, the one or more health-related or subject parameters may be one or more vital signs obtained for the subject, one or more laboratory results obtained for the subject, and/or one or more characteristics of the subject, such as e.g. age, gender, or demographic of the subject.

Non-limiting examples of health-related or subject parameters potentially indicated by or included in the set of subject data can include one or more of a subject age value, a subject heart rate value, a subject respiratory rate value, a subject diastolic blood pressure value, a subject systolic blood pressure value, a subject blood pressure value, a subject temperature value, at least one value related to Monocyte Distribution Width (MDW), a White Blood Cell Count (WBC), a subject oxygen saturation value, a lactate value, a partial pressure value of CO₂, a neutrophil value, and a Glasgow coma score of the subject. One or more of the parameters or corresponding parameter values indicated by the set of subject data can be measured or obtained based on a measurement, for example based on or using one or more samples from the subject, such as blood samples.

According to an embodiment, the subject data or set of subject data comprises at least one health-related or subject parameter of or associated with the subject, wherein analysing the subject data with respect to the one or more occult sepsis criteria comprises determining whether the at least one health-related parameter meets and/or fulfils at least one of the one or more occult sepsis criteria. Therein, analysing the subject data with respect to the one or more occult sepsis criteria can include determining one or more health-related parameters based on evaluating the subject data and comparing the one or more determined health-related parameters to the one or more occult sepsis criteria. Optionally, determining the one or more health-related parameters can include deriving said one or more health-related parameters from the set of subject data, for example based on computing or calculating said one or more health-related parameters and/or based on extracting said one or more health-related parameters from the set of subject data.

According to an embodiment, the one or more occult sepsis criteria include one or more of:
(a) a temperature equal to or above 36° C.;
(b) a temperature equal to or below 38° C.;
(c) a temperature between 36° C. and 38° C.;
(d) a systolic blood pressure equal to or above 90 mm Hg;
(e) a lactate value equal to or below 2 mmol/L;
(f) a respiratory rate equal to or below 20 breaths/minute;
(g) partial pressure of CO₂ equal to or above 32 mmHg;
(h) a heart rate equal to or below 90 bpm;
(i) a white blood cell count value equal to or above 4×10⁹/L;
(j) a white blood cell count value equal to or below 12×10⁹/L;
(k) a white blood cell count value between 4×10⁹/L and 12×10⁹/L;
(l) neutrophil bands equal to or below 10%, or equal to or below 10% immature forms of neutrophils;
(m) one or more complaints selected from the group consisting of fever, chills, rigors, cough, soft tissue, and infection;
(n) exclusion of Systemic Inflammatory Response Syndrome;
(o) a systolic blood pressure equal to or above 100 mmHg;
(p) a respiratory rate equal to or below 21 breaths/minute; and
(q) a Glasgow coma score equal to or above 14 and/or a Glasgow coma score equal to 15.

Therein, each of the occult sepsis criteria may be indicative of a risk for occult sepsis. An overall risk for occult sepsis may be given by or computed based on those occult sepsis criteria that are fulfilled or met by the set of subject data of a given subject, patient or individual. Further, the “temperature” in some of the occult sepsis criteria may refer to a “body temperature” of the subject. The term “neutrophil bands” (also referred to as “bands” or “leukocyte bands”) may refer to or can be synonymously used for “immature neutrophils”, which are a type of white blood cells or leukocytes. Moreover, the Glasgow coma score generally assesses a subject's level of responsiveness, wherein eye opening, verbal response and motor response are evaluated and combined in the overall Glasgow coma score.

Accordingly, the method may comprise analysing the set of subject data with respect to one or more of the occult sepsis criteria (a)-(q) listed above. Therein, it may be determined whether one or more of the occult sepsis criteria (a)-(q) are fulfilled or met by the set of subject data. This may include comparing the one or more health-related parameters indicated by or included in the set of subject data to one or more threshold values for one or more health-related parameters associated with one or more occult sepsis criteria. Accordingly, one or more occult sepsis criteria can be associated with one or more threshold values or threshold criteria for one or more health-related parameters indicated by or included in the set of subject data.

In an exemplary implementation, the method may comprise determining, based on analysing and/or evaluating the set of subject data, one or more health-related parameters comprising one or more of a temperature or body temperature of the subject, a blood pressure of the subject, a systolic blood pressure of the subject, a diastolic blood pressure of the subject, a lactate value of the subject, a respiratory rate of the subject, a partial pressure of CO₂ of the subject, a heart rate of the subject, a complaint of the subject, and a Glasgow coma score of the subject. The method may further comprise comparing the determined one or more health-related parameters of the subject to one or more threshold values for the one or more health-related parameters associated with the one or more occult sepsis criteria, for example thereby determining and/or coming to a conclusion whether said one or more occult sepsis criteria are fulfilled or met by the one or more health-related parameters.

According to an embodiment, analysing the set of subject data with respect to the one or more occult sepsis criteria comprises determining that at least occult sepsis criteria (a)-(c), at least occult sepsis criteria (a)-(d), at least occult sepsis criteria (a)-(d) and (f), at least occult sepsis criteria (a)-(d) and (f) and (k), at least occult sepsis criteria (a)-(d) and (f) and (k)-(l), at least occult sepsis criteria (a)-(d) and (f) and (k)-(m), at least occult sepsis criteria (a)-(f)), at least occult sepsis criteria (a)-(e), at least occult sepsis criteria (a)-(f), at least occult sepsis criteria (a)-(f) and (k), at least occult sepsis criteria (a)-(f) and (k)-(l), at least occult sepsis criteria (a)-(f) and (k)-(m), at least occult sepsis criteria (a)-(g), at least occult sepsis criteria (a)-(h), at least occult sepsis criteria (a)-(i), at least occult sepsis criteria (a)-(j), at least occult sepsis criteria (a)-(k), at least occult sepsis criteria (a)-(l), at least occult sepsis criteria (a)-(m), at least occult sepsis criteria (a)-(n), at least occult sepsis criteria (a)-(o), at least occult sepsis criteria (a)-(p), or occult sepsis criteria (a)-(q) are each fulfilled.

Accordingly, the method may comprise determining that at least a subset or plurality of the occult sepsis criteria (a)-(q) is each fulfilled or met by the set of subject data. Possible and non-limiting examples of subsets of occult sepsis criteria that can be analysed in terms of fulfilment by the set of subject data include:
Subset 1: occult sepsis criteria (a)-(c),
Subset 2: occult sepsis criteria (a)-(d),
Subset 3: occult sepsis criteria (a)-(d) and (f),
Subset 4: occult sepsis criteria (a)-(d) and (f) and (k),
Subset 5: occult sepsis criteria (a)-(d) and (f) and (k)-(l),
Subset 6: occult sepsis criteria (a)-(d) and (f) and (k)-(m),
Subset 7: occult sepsis criteria (a)-(f)),
Subset 8: occult sepsis criteria (a)-(e),
Subset 9: occult sepsis criteria (a)-(f),
Subset 10: occult sepsis criteria (a)-(f) and (k),
Subset 11: occult sepsis criteria (a)-(f) and (k)-(l),
Subset 12: occult sepsis criteria (a)-(f) and (k)-(m),
Subset 13: occult sepsis criteria (a)-(g),
Subset 14: occult sepsis criteria (a)-(h),
Subset 15: occult sepsis criteria (a)-(i),
Subset 16: occult sepsis criteria (a)-(j),
Subset 17: occult sepsis criteria (a)-(k),
Subset 18: occult sepsis criteria (a)-(l),
Subset 19: occult sepsis criteria (a)-(m),
Subset 20: occult sepsis criteria (a)-(n),
Subset 21: occult sepsis criteria (a)-(o), and
Subset 22: occult sepsis criteria (a)-(p).

The determination as to whether a plurality of occult sepsis criteria, all criteria of a subset of occult sepsis criteria or all occult sepsis criteria (a)-(q) are fulfilled may allow to reliably determine whether the subject is at risk for occult sepsis or has occult sepsis. In particular, a number of false positives and/or false negatives in the determination of occult sepsis may be reduced. In turn, an overall quality and robustness in the determination or assessment of occult sepsis can be increased.

According to an embodiment, analysing the set of subject data with respect to the one or more occult sepsis criteria comprises determining that at least occult sepsis (a)-(f) and (k)-(m) are each fulfilled. Alternatively or additionally, analysing the set of subject data with respect to the one or more occult sepsis criteria comprises determining that the aforementioned subset 12 of the occult sepsis criteria is fulfilled or met. The inventors have found that e.g. the cumulative fulfilment of occult sepsis criteria (a)-(f) and (k)-(m) or subset 12 is a reliable indicator for a high risk of occult sepsis at the subject and/or of the actual presence of occult sepsis at the subject. Hence, by determining that at least occult sepsis criteria (a)-(f) and (k)-(m) are each fulfilled, the number of false positives and/or false negatives in the determination of occult sepsis may be reduced, and the overall quality and robustness in the determination or assessment of occult sepsis can be further increased.

According to an embodiment, the method further comprises determining, based on analysing the set of subject data with respect to the one or more occult sepsis criteria, whether or not the subject is at risk for occult sepsis and/or whether or not the subject has occult sepsis. Accordingly, based on the analysis of the set of subject data with respect to the occult sepsis criteria, a binary result or binary classification result indicative of whether or not the subject has occult sepsis may be computed and/or generated. Such binary result can, for example, be indicated by a value of a binary flag. Accordingly, at least in certain instances, determining the risk of occult sepsis for a subject may mean or comprise determining whether or not the subject has occult sepsis and/or is likely to have or develop occult sepsis, e.g. within a predetermined period of time.

Alternatively or additionally, also a likelihood or probability for occult sepsis to occur at the subject, for example within a predetermined period of time, may be computed or calculated based on the analysis of the set of subject data with respect to the occult sepsis criteria. For instance, based on determining and/or computing a number of occult sepsis criteria (a)-(q) met or fulfilled, a likelihood or probability for occult sepsis to occur at the subject may be computed. Alternatively or additionally, the likelihood or probability for occult sepsis to occur at the subject may be computed based on determining which of subsets 1 to 22 of the occult sepsis criteria is fulfilled, wherein each of the subsets 1 to 22 may be associated with a particular likelihood or probability for occult sepsis to occur at the subject. Accordingly, at least in certain instances, determining the risk of occult sepsis for a subject may mean or comprise determining a probability or likelihood for occult sepsis to occur at the subject, e.g. within a predetermined period of time.

Alternatively or additionally, a level or tier of a risk for occult sepsis to occur at the subject, such as low, medium and high risk for occult sepsis, may be determined based on the analysis of the set of subject data with respect to the occult sepsis criteria. For example, such determination of the level or tier of the risk for occult sepsis may be based on determining and/or computing a number of occult sepsis criteria (a)-(q) met or fulfilled. Alternatively or additionally, a level or tier of a risk for occult sepsis to occur at the subject may be computed based on determining which of subsets 1 to 22 of the occult sepsis criteria is fulfilled, wherein each of the subsets 1 to 22 may be associated with a particular level or tier of a risk for occult sepsis to occur at the subject.

Optionally, a predefined metrics may be applied for evaluating the set of subject data with respect to the one or more occult sepsis criteria. For example, different weighting factors may be applied for at least some of the occult sepsis criteria (a)-(q). Any other metrics can be applied instead of or in addition thereto.

According to an embodiment, analysing the set of subject data with respect to the one or more occult sepsis criteria comprises predicting if the subject is having occult sepsis or not. This may include coming to a conclusion as to whether or not the subject has or is at risk of occult sepsis.

In an exemplary implementation, the method may comprise determining, based on evaluating and/or analysing the set of subject data with respect to the one or more occult sepsis criteria, whether or not the subject is at risk to develop occult sepsis and/or has occult sepsis. Alternatively or additionally, the method may comprise coming to a conclusion as to whether or not the subject has occult sepsis and/or is at risk of developing occult sepsis. Alternatively or additionally, the method may comprise obtaining and/or generating a binary result indicative of whether or not the subject has occult sepsis and/or is at risk to develop occult sepsis.

In an exemplary implementation, the method may comprise classifying the subject into at least two classes, for example at least a first class associated with subjects having occult sepsis and at least a second class of subjects not having occult sepsis. Also, more than two classes may be defined, wherein, for example, each class of the plurality of classes may correspond to or be associated with a particular level or tier of the occult sepsis risk, such as e.g. low, medium and high risk for occult sepsis.

For example, classifying may comprise assigning one of the plurality of classes to the subject and/or assigning the subject to one of the plurality of classes. Alternatively, classifying may include computing, for each class of a plurality of classes, a respective probability, said respective probability being indicative of a probability for the subject to belong to the respective class, and optionally selecting the class with the highest partial probability among the computed probabilities as the class to which the subject belongs or is classified into. The method may further comprise determining, computing and/or calculating a classification result indicative of the class the subject is classified into, and optionally indicative of the likelihood or probability that the subject belongs to the selected class. Such classification result can be a binary result or a classification result for more than two classes.

According to an embodiment, the method further comprises generating, upon determining that at least a part of the subject data and/or at least one health-related parameter comprised in the subject data meets at least one of the one or more occult sepsis criteria, a notification informative of the risk for the subject having occult sepsis, e.g., notification informative of the risk for the subject having occult sepsis within a predetermined period of time. The predetermined period of time may be between 0 hours and 48 hours, for example between 0 hours and 24 hours. Preferably the predetermined period of time may be between 0 hours and 12 hours or between 12 hours and 24 hours. It should be noted that “0 hours” may refer to or denote the time point of assessing occult sepsis, and/or, may refer to or denote the time point of carrying out the method of determining occult sepsis disclosed herein.

Optionally, the generated notification informative of the risk for the subject having occult sepsis may be stored at a data storage of the computing device or of an external computing device or system. For example, the notification or information corresponding thereto may be stored in an electronic health record associated with the subject.

The “notification informative of the risk for the subject having occult sepsis” may refer to a notification whether or not the subject is at risk for occult sepsis, respectively, whether or not the subject has occult sepsis or is likely to have occult sepsis. Accordingly, the notification may contain or be indicative of a binary result as to whether or not the subject has occult sepsis. Alternatively or additionally, the notification may include a likelihood or probability for occult sepsis to occur at the subject. Alternatively or additionally, the notification may include a level or tier of the occult sepsis risk, such as e.g. low, medium and high risk for occult sepsis.

Generally, by generating the notification informative of the risk for the subject having occult sepsis, HCPs may be reliably informed about a potential occult sepsis being present or being developed at the subject, optionally within the predetermined period of time. This may enable HCPs to take appropriate actions, for example to initiate an appropriate therapy, to keep the subject under surveillance and/or to perform one or more measurements for one or more health-related parameters. Also, it may be avoided that a subject having only subtle or no symptoms of sepsis is erroneously discharged from an emergency department, clinic or hospital. As a consequence, delays in the treatment can be effectively avoided and a timely treatment and therapy can be ensured.

According to an embodiment, the notification is notifiable to a medical practitioner or HCP via a user interface of a computing device. Alternatively or additionally, the method may comprise outputting and/or providing the notification at the user interface of the computing device. By outputting the notification at the user interface, a subject potentially having occult sepsis may be highlighted for the HCP or medical practitioner. For example, the notification may be designed or configured to stand out from a number of other subject information shown or output at the user interface to direct an HCPs attention to the potential risk for occult sepsis at the given subject, e.g. even though the subject may have no or only subtle symptoms of sepsis.

Accordingly, the computing device may comprise a user interface configured to notify and/or output the notification informative of the risk for occult sepsis. For instance, the user interface can be configured to notify or output the notification based on one or more of text, sound, haptic and visual information. Accordingly, the notification can include one or more of a text-based notification, a sound-based notification, a haptic notification, and a visual notification.

According to an embodiment, the notification includes a recommendation or instruction to order one or more of a lactate test, antibiotic treatment for the subject, one or more further tests for assessing sepsis risk, a Procalcitonin test, and a Monocyte Distribution Width (MDW) test. Instructing or recommending to order one or more additional tests may allow to validate or confirm a previously determined risk for occult sepsis, for example based on re-analysing the set of subject data supplemented by the one or more additional tests with respect to the one or more occult sepsis criteria. Also, in some examples, an erroneously determined high risk for occult sepsis may be detected based on re-analysing the set of subject data supplemented by the one or more additional tests with respect to the one or more occult sepsis criteria.

According to an embodiment, confirming that the obtained sepsis score meets the at least one threshold value for the sepsis score comprises:
comparing the obtained at least one sepsis score to the at least one threshold value for the sepsis score; and
determining that the obtained at least one sepsis score reaches, exceeds or falls below the at least one threshold value for the sepsis score.
   As mentioned above, depending on the scale of the sepsis score chosen, it may be determined whether the sepsis score reaches, exceeds or falls below a particular threshold value for the sepsis score. For instance, when the sepsis score is given on a positive scale, it may be determined whether the sepsis score reaches and/or exceeds the threshold value. On the other hand, when the sepsis score is given on a negative scale, it may be determined whether the sepsis score reaches and/or falls below the threshold value.

According to an embodiment, the at least one sepsis score is a numerical measure and/or continuous variable indicative of the risk, likelihood and/or probability for the subject developing the sepsis event, optionally within a predetermined period of time. Therein, the sepsis score may be provided on an arbitrary scale and/or range from a minimum value, for example zero or 0, to a maximum value, for example one, 10 or 100. Any other scale, including relative and absolute scales, can be used instead to represent the sepsis score.

According to an embodiment, the at least one threshold value for the sepsis score is at least 40% of the maximum sepsis score value, at least 50% of the maximum sepsis score value, or at least 60% of the maximum sepsis score value, or at least 70% of the maximum sepsis score value, or at least 80% of the maximum sepsis score value, or at least 90% of the maximum sepsis score value. Hence, the threshold value for the sepsis score may be chosen or selected to indicate or reflect medium to high sepsis scores, thereby indicating a considerable risk for sepsis. Such medium to high sepsis score in conjunction with fulfilment of one or more of the occult sepsis criteria may provide for a reliable indicator for occult sepsis. Hence, by selecting an appropriate threshold value for the sepsis score and evaluating the set of subject data with respect to the one or more occult sepsis criteria, occult sepsis can be reliably detected at the subject.

As mentioned above, the sepsis score may be provided in the form of a particular level or tier for the sepsis risk. In this example, the threshold value for the sepsis score may indicate one or more levels or tiers, which the level or tier indicated by the sepsis score should match. For instance, the sepsis score may be provided as low, medium or high risk for sepsis, and the threshold value for the sepsis score may correspond to “at least medium” or “at least high” risk for sepsis.

According to an embodiment, obtaining the sepsis score may comprise accessing and/or retrieving the sepsis score e.g. from at least one memory or data storage of the computing device that carries out the method of the present disclosure, from the memory or data storage of another computing device, or from another remote data storage, such as e.g. a database, a secondary memory, a cloud storage or the like. Further, retrieving the sepsis score may comprise downloading the sepsis score. Additionally or alternatively, obtaining the sepsis score may comprise receiving the sepsis score, e.g. from a user or a computing device different from the computing device accessing the sepsis score. The two options are not mutually exclusive. For instance, obtaining the sepsis score may comprise receiving the sepsis score, storing the sepsis score in the memory of the computing device and retrieving the sepsis score by accessing said memory.

According to an embodiment, obtaining the at least one sepsis score comprises determining, based on at least a part of the set of subject data and/or based on supplementary subject data, the at least one sepsis score. In other words, the at least sepsis score may be determined based on or using at least a part of the subject data and/or based on or using supplementary subject data. Therein, the part of the subject data used to determine the sepsis score may at least partly overlap with and/or may at least partly differ from the subject data that is analysed with respect to the occult sepsis criteria to determine whether or not the subject is at risk for occult sepsis. For instance, the part of the subject data used to determine the sepsis score may overlap with the subject data that is analysed with respect to the occult sepsis criteria if they have a date in common and/may differ therefrom if at least one parameters is not shared among the data sets. Alternatively or additionally, the supplementary subject data used to determine the sepsis score may at least partly overlap with and/or may at least partly differ from the subject data that is analysed with respect to the occult sepsis criteria to determine whether or not the subject is at risk for occult sepsis.

Generally, determining the sepsis score can advantageously allow for a quantitative assignment of a health risk or risk status to the subject, such as for example a low, medium or high risk for sepsis. In particular, a number of false positives and/or a number of false negatives in determining or assessing whether a subject has a low, medium or high risk for sepsis, can be advantageously reduced by means of the computer-implemented method described herein.

As used herein, determining the sepsis score may include computing and/or assessing the risk for sepsis. For instance, determining the sepsis risk or sepsis score may generally relate to or include finding out or coming to a decision about the risk for sepsis. Determining the sepsis risk or sepsis score may optionally include a corresponding reasoning or assessment of the risk, a calculation of the risk or sepsis score and/or a computation of the risk or sepsis score. Alternatively or additionally, computing the risk for sepsis may include determining based on mathematical means, algorithms and/or calculations, the risk for sepsis. In particular the calculations may be computer-aided, computer-assisted and/or computer-implemented. For example, determining the sepsis risk or sepsis score may be carried out by using a determination algorithm. In particular, the determination algorithm can be configured to receive and process input data, the input data comprising and/or being based on the set of patient data. The determination algorithm can be configured to generate output data, the output data being indicative of the sepsis risk or the sepsis score. Alternatively or additionally, assessing the sepsis risk may include determining an importance, a significance, a value, a level and/or a tier for the risk for sepsis and/or the sepsis score.

In an exemplary embodiment, the method comprises obtaining the at least part of the subject data and/or the supplementary subject data, based on which the sepsis score can be determined. Therein, obtaining said at least part of the subject data and/or the supplementary subject data may comprise accessing said data and/or retrieving the data e.g. from at least one memory or data storage of the computing device that carries out the method of the present disclosure, from the memory or data storage of another computing device, or from another remote data storage, such as e.g. a database, a secondary memory, a cloud storage or the like. Further, retrieving the at least part of the subject data and/or the supplementary subject data may comprise downloading said data. Additionally or alternatively, obtaining the at least part of the subject data and/or the supplementary subject data may comprise receiving said data, e.g. from a user or a computing device different from the computing device accessing the data. The two options are not mutually exclusive. For instance, obtaining the at least part of the subject data and/or the supplementary subject data may comprise receiving said data, storing said data in the memory of the computer device and retrieving the data by accessing said memory.

The method according to the present disclosure can comprise determining at least one sepsis score indicative of the risk for a sepsis event or indicative of the risk of sepsis occurring at the patient or subject. In particular, determining the at least one sepsis score can include computing the risk for sepsis occurring at the subject based on, e.g. by using, at least one machine learning (ML) model, e.g. a predictive ML model. For example, the ML model can be configured to compute a numeric sepsis risk score by using subject demographic (such as age or gender) and subject clinical data, which may be comprised in the at least part of the subject data and/or the supplementary subject data that is used to determine the sepsis score. Subject clinical data may include values of one or more subject vital signs and/or laboratory results obtained from an electronic health record (EHR) of or associated with the subject.

In an example, the machine learning model implemented at the computing device and optionally used to compute or determine the sepsis score can be configured to receive and process input data, the input data comprising and/or being based on at least a part of the subject data and/or the supplementary subject data. For instance, the determination algorithm mentioned above may comprise or consist of the ML model. In other words, determining the at least one sepsis score may include computing the risk for the subject developing the sepsis event based on at least one machine learning model, wherein the at least one machine learning model may be configured to receive and process input data, the input data comprising and/or being based on at least a part of the subject data and/or the supplementary subject data.

The at least one machine learning model can, for example, comprise a trained gradient boosting algorithm, a trained artificial neural network, a trained feed forward neural network, a trained convolutional neural network and/or a trained deep neural network.

According to an embodiment, confirming that the sepsis score meets the threshold value for the sepsis score is carried out by means of or implemented as a rule-based algorithm. Alternatively or additionally, set of subject data may be analysed with respect to the one or more occult sepsis criteria by means of the rule-based algorithm. For instance, obtaining and/or determining the sepsis score, e.g. using a ML model, may trigger the rule-based algorithm to determine whether and/or to confirm that the sepsis score meets the threshold value for the sepsis score, and optionally may trigger the rule-based algorithm to evaluate and/or analyse the set of subject data with respect to the one or more occult sepsis criteria. Alternatively, confirming that the sepsis score meets the threshold value for the sepsis score and/or analysing the set of subject data with respect to the one or more occult sepsis criteria may be implemented by means of a further ML model, which may differ from the ML model used to determine and/or compute the sepsis score.

According to an embodiment, the at least part of the subject data and/or the supplementary subject data set utilised to determine the sepsis score includes and/or is indicative of one of, a plurality of, or all of: a subject age value, a subject heart rate value, a subject respiratory rate value, a subject diastolic blood pressure value, a subject systolic blood pressure value, a blood pressure value, a subject temperature value, at least one value related to Monocyte Distribution Width (MDW), a White Blood Cell Count (WBC), a subject oxygen saturation value, a lactate value, a partial pressure of CO₂, a neutrophil value, a neutrophil band value, an indication about one or more complaints, and a Glasgow coma. By determining the sepsis score and/or the risk for sepsis based on or using one or more of the aforementioned health-related parameters or subject parameters, a health state of the subject can be comprehensively examined or evaluated. As a consequence, the determination of the risk for sepsis and/or the sepsis score can be individualized to individual subjects or subject sub-groups, which can further reduce the number of false positives and/or false negatives. Also, the time required for determining the risk of sepsis and/or the sepsis score can be significantly reduced.

In an example, the risk for sepsis and/or the sepsis score may be determined based on or using at least one value related MDW and at least one of a subject age value, a subject heart rate value, a subject respiratory rate value, a subject diastolic blood pressure value, a subject systolic blood pressure value, a subject temperature value and a subject oxygen saturation value. Using at least one value of or related to MDW and at least one of the aforementioned further health-related parameters to compute or determine the sepsis score can allow to accurately determine the risk for sepsis. Hence, quality and accuracy of the determined sepsis score can be increased.

It should be noted that “a parameter related to MDW” may be referred to herein as MDW value. Further, “a value related to MDW” in the context or the present disclosure can refer to a parameter indicative of the monocyte cell population of the subject's blood and/or containing information about or related to MDW. MDW or the MDW value may be indicative of a variation or dispersion of the monocyte cell size within the monocyte cell population of the subject's blood. Such variation or dispersion in the size of monocyte blood cells can be obtained by various measurements, including measurements based on the Coulter principle, flowcytometry, fluorescence, light scattering measurements and others.

For instance, when a complete blood count (CBC) is obtained from a blood sample, using an analyser such as a haematology analyser on the same blood sample may provide data about a subpopulation of cells that is much richer than simply a count or proportion of those cells compared to other subpopulations of cells within a sample. For example, a monocyte cell population parameter that reflects monocyte activation may be obtained. One such monocyte parameter is monocyte distribution width (referred to herein as MDW, and also referred to as monocyte anisocytosis). MDW represents the volume distribution of the monocyte population in a blood sample; thus, this morphometric parameter reflects variability in monocyte cell volume.

Morphological changes in monocyte cell volume can occur early as a result of pathogen recognition-induced monocyte activation, and thus MDW can be altered early in disease trajectory. MDW has demonstrated capability in identification of patients with sepsis in high-risk populations (Crouser et al. Crit Care Med. 2019;47(8):1018-1025; Crouser et al. Chest. 2017;152(3):518-526; Crouser et al. Intensive Care; 2020;8:33).

In particular, MDW can be regarded a morphometric leukocyte biomarker that can signify the standard deviation in the width distribution of monocytes, a leukocyte critical to the initiation of the innate immune response, and an early indicator of infection.

According to an embodiment, the at least part of the subject data and/or the supplementary subject data set utilised to determine the sepsis score is indicative of and/or includes at least one of: a plurality of subject heart rate values, a plurality of subject respiratory rate values, a plurality of subject diastolic blood pressure values, a plurality of subject systolic blood pressure values, a plurality of subject temperature values, a plurality of subject oxygen saturation values, a plurality of values related to MDW, and a plurality of subject WBC values.

According to the present disclosure, the plurality of values of a subject or health-related parameter may be obtained at the same or different time points, e.g. at different time points within a time interval. In particular, values of a plurality of values of a health-related parameter, such as a plurality of heart rate values, may be obtained at different time points, thereby allowing, for instance, taking into account the variation of the parameter over time and the effect and/or impact of said variation on the computed sepsis score. For instance, each value of a plurality of values of a subject or health-related parameter is obtained at respective time points, in such a way that, for each pair of values of said plurality of values, the time point at which the first value of that pair of values is obtained, is different from the time point at which the second value of that pair of values is obtained.

In an example, corresponding time information for each value of the plurality of values of a subject or health-related parameter may be included in the at least part of the subject data and/or the supplementary subject data set utilised to determine the sepsis score, for example in the form of a timestamp associated with the respective value. The timestamps or time information of different values of said plurality of values may be identical, for example when two measurements of a parameter have been performed at the same time, concurrently and/or at similar times, or may differ, for example when two consecutive measurements of a parameter have been made.

In an exemplary embodiment, the at least part of the subject data and/or the supplementary subject data set utilised to determine the sepsis score can be indicative of the plurality of subject heart rate values and determining the at least one sepsis score can be based on a maximum value of the subject heart rate, a minimum value of the subject hearth rate, a most recent value of the subject heart rate and/or one or more mean values of the subject heart rate. Alternatively or additionally the at least part of the subject data and/or the supplementary subject data set utilised to determine the sepsis score can be indicative of the plurality of subject respiratory rate values and determining the at least one sepsis score can be based on a maximum value of the subject respiratory rate, a minimum value of the subject respiratory rate, a most recent value of the subject respiratory rate and/or one or more mean values of the subject respiratory rate. Alternatively or additionally, the at least part of the subject data and/or the supplementary subject data set utilised to determine the sepsis score can be indicative of the plurality of MDW values and determining the at least one sepsis score can be based on a maximum value of MDW, a minimum value of MDW, a most recent value of MDW and/or one or more mean values of the MDW. Alternatively or additionally, the at least part of the subject data and/or the supplementary subject data set utilised to determine the sepsis score can be indicative of the plurality of subject diastolic blood pressure values and determining the at least one sepsis score can be based on a maximum value of the subject diastolic blood pressure, a minimum value of the subject diastolic blood pressure, a most recent value of the subject diastolic blood pressure and/or one or more mean values of the subject diastolic blood pressure. Alternatively or additionally, the at least part of the subject data and/or the supplementary subject data set utilised to determine the sepsis score can be indicative of the plurality of subject systolic blood pressure values and determining the at least one sepsis score can be based on a maximum value of the subject systolic blood pressure, a minimum value of the subject systolic blood pressure, a most recent value of the subject systolic blood pressure and/or one or more mean values of the subject systolic blood pressure. Alternatively or additionally, the at least part of the subject data and/or the supplementary subject data set utilised to determine the sepsis score can be indicative of the plurality of subject oxygen saturation values and determining the at least one sepsis score can be based on a maximum value of the subject oxygen saturation, a minimum value of the subject oxygen saturation, a most recent value of the subject oxygen saturation and/or one or more mean values of the subject oxygen saturation. Alternatively or additionally, the at least part of the subject data and/or the supplementary subject data set utilised to determine the sepsis score can be indicative of the plurality of subject WBC values and determining the at least one sepsis score can be based on a maximum value of the subject WBC, a minimum value of the subject WBC, a most recent value of the subject WBC, and/or one or more mean values of the subject WBC. Alternatively or additionally, the at least part of the subject data and/or the supplementary subject data set utilised to determine the sepsis score can be indicative of the plurality of subject temperature values and determining the at least one sepsis score can be based on a maximum value of the subject temperature, a minimum value of the subject temperature, a most recent value of the subject temperature and/or one or more mean values of the subject temperature.

Accordingly, any of the maximum parameter value(s), the minimum parameter value(s), the most recent value(s) and/or the mean value(s) can be computed by the computing device and used to determine the sepsis score. In particular, this can allow for considering variation over time, such as the evolution or the development of one or more of the aforementioned subject parameters for determining the sepsis score over time. In particular, the sepsis score may be updated upon receiving one or more further parameter values of one or more health-related or subject parameters described herein as being usable for determining the sepsis score.

Based on the obtained or received at least part of the subject data and/or the supplementary subject data set utilised to determine the sepsis score, one or more of the aforementioned health-related parameter values may be computed, determined and/or selected by the computing device. Accordingly, the method may comprise determining, computing and/or selecting a minimum value, a maximum value, a most recent value and/or a mean value among a plurality of parameter values of a particular parameter contained in the at least part of the subject data and/or among the supplementary subject data set utilised to determine the sepsis score. For example, the method may comprise determining, computing and/or selecting a minimum value, a maximum value, a most recent value and/or a mean value among the plurality of subject heart rate values, among the plurality of subject respiratory rate values, among the plurality of subject diastolic blood pressure values, among the plurality of values related to MDW, among the plurality of subject systolic blood pressure values, among the plurality of subject temperature values, among the plurality of subject oxygen saturation values, and/or among the plurality of subject WBC values. The same applies to any other parameter described herein as being potentially usable for computing the sepsis score. Alternatively or additionally, one or more of these parameter values, i.e. the minimum value, the maximum value, the most recent value and/or the mean value of a plurality of parameter values of a particular parameter, may be received as the at least part of the subject data and/or the supplementary subject data set utilised to determine the sepsis score at the computing device and used as input data to compute the sepsis score.

In an embodiment, the method further comprises determining, based on the at least one sepsis score, a sepsis risk level or risk tier. In particular, determining the sepsis risk level or risk tier comprises selecting the sepsis risk level or risk tier from a plurality of risk levels nor risk tiers. Selecting a risk level or tier may include identifying a risk level or tier that matches, corresponds to or is associated with the determined sepsis score. Determining the sepsis risk level or tier may further improve risk stratification based on the computed sepsis score and may simplify interpretation thereof, for example by a clinician.

For instance, selecting the sepsis risk level from the plurality of risk levels may comprises comparing the at least one sepsis score with one or more risk score thresholds.

In an example, the plurality of risk levels can comprise a first risk level, a second risk level and a third risk level, wherein if the at least one sepsis score is lower than a first threshold, the first risk level is selected as sepsis risk level, if the at least one sepsis score is greater than the first threshold and lower than a second threshold, the second risk level is selected as sepsis risk level, and if the at least one sepsis score is greater than the second threshold, the third risk level is selected as sepsis risk level. The first, second and third risk level may respectively correspond to a low. Medium and high risk for sepsis.

The sepsis risk score can also be used to trigger active notifications such as © popup alerts. To facilitate interpretation, the risk score or sepsis score can be stratified into a plurality of risk levels or tiers, for example three risk tiers—low, medium, and high risk. Herein risk tiers can also referred to as “risk levels”. Alternatively, more than three or only two risk tiers can be used.

Alternatively or additionally, selection and/or determination of the risk level may be carried out by using an ML model which is trained to select and/or determine the risk level by using the input data. In this case, the risk level may be provided directly as output of the ML model. Accordingly, an output of the computing device may include an indication or information indicative of the risk level or risk tier, to which the subject is associated, according to or based on the determined sepsis score.

In another embodiment, the at least part of the subject data and/or the supplementary subject data set utilised to determine the sepsis score comprises, e.g. consists of, the subject age value, the subject heart rate value, the subject respiratory rate value, the subject diastolic blood pressure value, the subject systolic blood pressure value, the subject temperature value, the subject oxygen saturation value and optionally the subject WBC value. Using all these parameters and optionally WBC may further improve accuracy of the determined sepsis score. In particular a plurality of parameter values for each of the aforementioned subject parameters, i.e. of the subject age, the subject heart rate, the subject respiratory rate, the subject diastolic blood pressure, the subject systolic blood pressure, the subject temperature, the subject oxygen saturation and optionally the subject WBC, may be included in the at least part of the subject data and/or the supplementary subject data set utilised to determine the sepsis score, as described above.

In another embodiment, the at least part of the subject data and/or the supplementary subject data set utilised to determine the sepsis score comprises, e.g. consists of, the subject heart rate value, the subject respiratory rate value, the subject diastolic blood pressure value, the subject systolic blood pressure value, the subject temperature value and the subject oxygen saturation value. Using all these parameters may further improve accuracy of the determined sepsis score. In particular, a plurality of parameter values for each of the aforementioned subject parameters, i.e. the subject heart rate, the subject respiratory rate, the subject diastolic blood pressure, the subject systolic blood pressure, the patient temperature and the patient oxygen saturation may be included in the at least part of the subject data and/or the supplementary subject data set utilised to determine the sepsis score, as described above.

According to a second aspect of the present disclosure, there is provided a computing device or system including one or more processors for data processing, wherein the computing device is configured to carry out steps of the method according to the first aspect of the present disclosure, as described hereinabove and hereinbelow.

The computing device may optionally comprise a data storage, for example storing at least a part of the set of subject data, data derived therefrom, the at least one sepsis score, one or more threshold values for the sepsis score and/or information or data related the determination of the risk of occult sepsis, such as the one or more occult sepsis criterial. Alternatively or additionally, software instructions or one or more computer programs comprising software instructions may be stored at the data storage, which, when executed by one or more processors of the computing device, may instruct the computing device to perform steps of the method described hereinabove and hereinbelow.

According to an embodiment, the computing device includes one or more machine learning models, for example implemented in a classifier circuitry or logic of the computing device.

The computing device described herein may refer to any data processing device, including a standalone computing device or server and computing networks with a plurality of inter-operating computing devices, such as a cloud computing system or server system. Alternatively or additionally, the computing device may be embodied, at least in part, as mobile device, such as a smart phone a tablet computer, a notebook or the like.

The computing device may, in an example, comprise one or more communication interfaces configured to communicate with one or more remote devices, for example an external data storage or database, and/or one or more remote computing devices. For instance, at least a part of the set of subject data and/or supplementary subject data may be received via the communication interface from an external data storage. Alternatively or additionally, the computed at least one sepsis score and/or information related to the determination of the risk for occult sepsis may be transmitted to one or more remote devices or stored at the external data storage.

The computing device may further comprise one or more user interfaces configured to control the computing device and/or to output information, such as the notification informative of the risk for occult sepsis, the sepsis score, the subject data, supplementary subject data, or other information or data.

According to a third aspect of the present disclosure, there is provided a computer program comprising instructions, which, when the program is executed by a computing device, cause the computing device to carry out the steps of the method according to the first aspect of the present disclosure, as described hereinabove and hereinbelow.

A fourth aspect of the present disclosure relates to a computer-readable medium, for example a non-transitory computer-readable medium, storing the computer program according to third aspect of the present disclosure.

These and other aspects of the disclosure will be apparent from and elucidated with reference to the appended figures, which may represent exemplary embodiments.

### BRIEF DESCRIPTION OF THE DRAWINGS

The subject-matter of the present disclosure will be explained in more detail in the following with reference to exemplary embodiments which are illustrated in the attached drawings, wherein:

FIG. 1 shows a computing device or system for determining, for a subject, a risk of having an occult sepsis according to an exemplary embodiment;

FIG. 2 shows an user interface of the computing device of FIG. 1; and

FIG. 3 shows a flow chart illustrating a method of determining, for a subject, a risk of having an occult sepsis according to an exemplary embodiment.

The figures are schematic only and not true to scale. In principle, identical or like parts are provided with identical or like reference symbols in the figures.

### DETAILED DESCRIPTION OF THE INVENTION

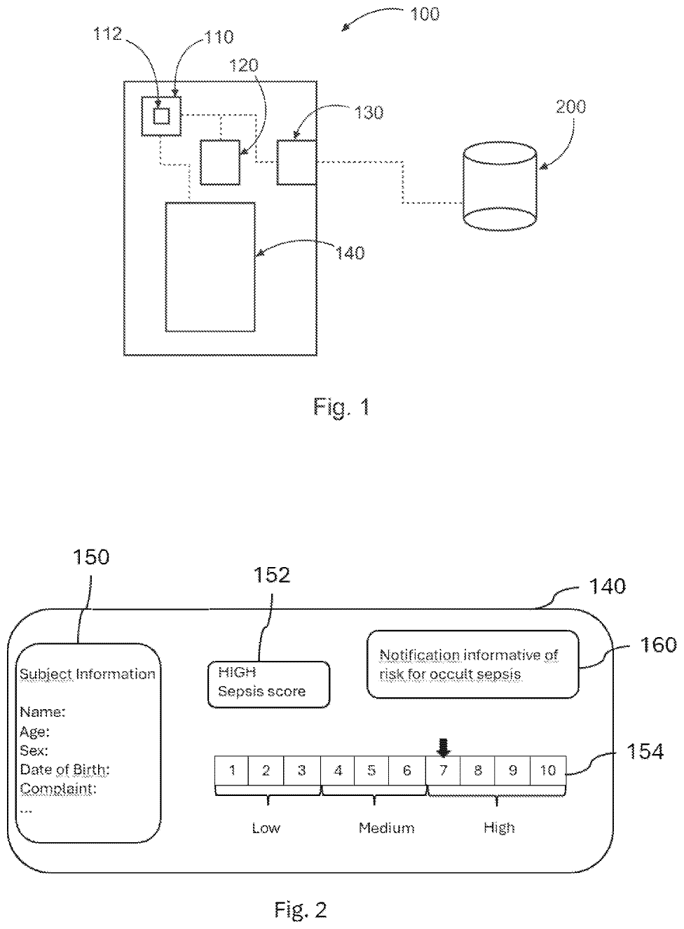

FIG. 1 shows a computing device or system 100, for example a clinical decision support system 100, configured to determine, for a subject, patient and/or individual, a risk of having an occult sepsis according to an exemplary embodiment. Alternatively or additionally, the computing device 100 may be configured to determine a patient-or subject-specific risk for occult sepsis.

The computing device 100 comprises a processing circuitry 110 or control circuitry 110 with one or more processors 112 for data processing. The computing device 100 further comprises at least one data storage 120 for storing data, one or more computer programs and/or corresponding software instructions (also referred to herein as “instructions”). For example, at least a part of a set of subject data indicative of a health state of the subject and/or indicative of one or more health-related parameters associated with the subject may be stored at the data storage 120. Alternatively or additionally, supplementary subject data, for example usable to determine a sepsis score, may be stored at the data storage 120.

The exemplary computing device 100 of FIG. 1 further comprises at least one communication circuitry or communication interface 130 for communicatively coupling the computing device 100 to one or more external data sources 200 and/or external computing devices 200 that may optionally store data and/or provide data to the computing device 100. The communication circuitry 130 may be configured for wired or wireless communication with the at least one external data source 200. It should be noted that the computing device 100 may comprise a plurality of communication circuits 130 or interfaces 130 for communicatively coupling the computing device 100 to a plurality of different external data sources or computing devices 200.

The one or more external data sources or computing devices 200 may for example be associated with one or more external servers communicatively coupled to the computing device 100, for example via the Internet, a LAN connection, a wireless connection or a wired connection. For example, the computing device 100 may be communicatively couplable to a hospital information system, a laboratory information system, a server of a health care provider, or any other server or data processing device.

The computing device 100 further includes a user interface 140 for receiving one or more user inputs and/or for providing or outputting information or data to the user. The user interface 140 may, for example, include one or more of a keyboard or mouse for controlling the computing device 100, a display for outputting graphical or visual information, a speaker for outputting audio information, and a haptic element for outputting haptic information. For example, the computing device 100 may be configured to display the at least one sepsis score at the user interface 140. Alternatively or additionally, the computing device 100 may be configured to output or display generated information or a notification 160 indicative of a risk of having occult sepsis at the user interface 140, as shown in FIG. 2.

As discussed in detail hereinabove and hereinbelow, the computing device 100 may be configured to determine, for a subject, a risk of having occult sepsis. Therein, the computing device 100 is configured to obtaining at least one sepsis score indicative of a risk of the subject developing a sepsis event. This may include accessing the at least one sepsis score at the data storage 120 or the one or more external data sources 200. Alternatively or additionally, this may include retrieving the at least one sepsis score from the data storage 120 or one or more external data sources 200. Alternatively or additionally, this may include one or more of computing, determining and/or calculating the at least one sepsis score, for example based on at least a part of the set of subject data and/or supplementary subject data.

The computing device 100 may further be configured to determine that the obtained at least one sepsis score meets at least one threshold value for the sepsis score. Upon or in response to confirming that the at least one sepsis score meets the at least one threshold for the sepsis score, the computing device 100 may analyse and/or evaluate a set of subject data indicative of a health state of the subject with respect to one or more occult sepsis criteria, which one or more occult sepsis criteria are indicative of a risk of the subject having an occult sepsis. Therein, the computing device 100 may be configured to determine whether at least one health-related parameter indicated by the set of subject data meets or fulfills one or more occult sepsis criteria.

The one or more occult sepsis criteria can include one or more of the following:
(a) a temperature equal to or above 36° C.;
(b) a temperature equal to or below 38° C. (c) a temperature between 36° C. and 38° C.;
(d) a systolic blood pressure equal to or above 90 mm Hg;
(e) a lactate value equal to or below 2 mmol/L;
(f) a respiratory rate equal to or below 20 breaths/minute;
(g) partial pressure of CO₂ equal to or above 32 mmHg;
(h) a heart rate equal to or below 90 bpm;
(i) a white blood cell count value equal to or above 4×10⁹/L;
(j) a white blood cell count value equal to or below 12×10⁹/L;
(k) a white blood cell count value between 4×10⁹/L and 12×10⁹/L;
(l) neutrophil bands equal to or below 10%, or equal to or below 10% immature forms of neutrophils;
(m) one or more complaints selected from the group consisting of fever, chills, rigors, cough, soft tissue, and infection;
(n) exclusion of Systemic Inflammatory Response Syndrome;
(o) a systolic blood pressure equal to or above 100 mmHg;
(p) a respiratory rate equal to or below 21 breaths/minute; and
(q) a Glasgow coma score equal to or above 14 and/or a Glasgow coma score equal to or above 15.

In particular, the computing device 100 may be configured to determine that at least a subset, a plurality of or all occult sepsis criteria (a)-(q) are each fulfilled or met by the set of subject data. In an example, the computing device 100 may be configured to determine that at least occult sepsis (a)-(f) and (k)-(m) are each fulfilled. However, the computing device 100 may alternatively be configured to determine that another subset or all of occult sepsis criteria (a)-(q) are each fulfilled, as described in detail in the summary section of the present disclosure.

Based on or by analysing the set of subject data with respect to the one or more occult sepsis criteria, the computing device 100 may determine whether or not the subject is at risk for occult sepsis and/or whether or not the subject has occult sepsis. Accordingly, based on the analysis of the set of subject data with respect to the occult sepsis criteria, a binary result indicative of whether or not the subject has occult sepsis may be computed and/or generated. Such binary result can, for example, be indicated by or stored in the form of a value of a binary flag at the data storage 120 of the computing device 100. Further, such binary result may be notified to, provided to, and/or brought to the attention of a user of the computing device 100, e.g. a medical practitioner or HCP, for example in the form of a notification 160 (see FIG. 2) informative of the risk for the subject having occult sepsis. In particular, the notification 160 may be displayed on the user interface 140.

Alternatively or additionally, the computing device 100 may compute or calculate, based on the analysis of the set of subject data with respect to the occult sepsis criteria, a likelihood or probability for occult sepsis to occur at the subject, for example within a predetermined period of time. For instance, based on determining and/or computing a number of occult sepsis criteria (a)-(q) met or fulfilled, a likelihood or probability for occult sepsis to occur at the subject may be computed. Optionally, the computed likelihood or probability may be notified to, provided to, and/or brought to the attention of a user of the computing device 100, e.g. a medical practitioner or HCP, for example in the form of a notification 160 (see FIG. 2) informative of the risk, likelihood or probability for the subject having occult sepsis.

Alternatively or additionally, the computing device 100 may compute or calculate, based on the analysis of the set of subject data with respect to the occult sepsis criteria, a level or tier of a risk for occult sepsis to occur at the subject, such as low, medium and high risk for occult sepsis. For example, such determination of the level or tier of the risk for occult sepsis may be based on determining and/or computing a number of occult sepsis criteria (a)-(q) met or fulfilled. Optionally, the computed level or tier of a risk for occult sepsis may be notified to, provided to, and/or brought to the attention of a user of the computing device 100, e.g. a medical practitioner or HCP, for example in the form of a notification 160 (see FIG. 2) informative of the level or tier of a risk for occult sepsis.

Optionally, the computing device 100 may apply a predefined metrics for analysing the set of subject data with respect to the one or more occult sepsis criteria. For example, different weighting factors may be applied for at least some of the occult sepsis criteria (a)-(q). However, any other metrics may be applied instead of or in addition thereto.

As mentioned above, the computing device 100 can be configured to generate, upon determining that at least a part of the subject data and/or at least one health-related parameter comprised in the subject data meets at least one of the one or more occult sepsis criteria, a notification 160 informative of the risk for the subject having occult sepsis, optionally within a predetermined period of time. The generated notification may be provided or output at the user interface 140 of the computing device 100. Alternatively or additionally, the computing device 100 may store the notification at its data storage 120 and/or at one or more external data sources 200. For example, the notification or information corresponding thereto may be stored in an electronic health record associated with the subject.

An exemplary notification 160 generated by the computing device 100 is shown in FIG. 2. In particular, FIG. 2 shows an exemplary output on the user interface 140 of the computing device 100 of FIG. 1.

As shown in FIG. 2, subject or patient information 150 can be displayed at the user interface 140, for example including information about one or more health-related or subject parameters, such as age, sex, complaints etc. can be displayed.

Moreover, information 152 related to the determined sepsis score can be displayed at the user interface 140. In the example shown in FIG. 2, the determined level or tier of the sepsis risk, i.e. “HIGH”, is displayed as information 152 related to the sepsis score.

Further, the numerical value of the sepsis score can be displayed, e.g. on as scale 154 or by other means 154. In the example of FIG. 2, the sepsis score ranges from a minimum value of 7 to a maximum value of 10, wherein for the exemplary subject a sepsis score of seven has been obtained, computed or calculated.

Further, the user interface 140 can display the notification 160 informative of the risk for occult sepsis. For instance, the notification 160 can be displayed in the form of a popup-alert or the like.

The notification 160 informative of the risk for the subject having occult sepsis can indicate whether or not the subject is at risk for occult sepsis, respectively, whether or not the subject has occult sepsis or is likely to have occult sepsis. Accordingly, the notification 160 may contain or be indicative of a binary result as to whether or not the subject has occult sepsis. Alternatively or additionally, the notification 160 may include a likelihood or probability for occult sepsis to occur at the subject. Alternatively or additionally, the notification 160 may include a level or tier of the occult sepsis risk, such as e.g. low, medium and high risk for occult sepsis.

Optionally, the computing device 100 may be configured to determine a recommendation and/or instruction for HCPs or medical practitioners, which recommendation or instruction can be displayed as part of the notification 160. The recommendation or instruction can indicate to HCPs or medical practitioners to order one or more of a lactate test, antibiotic treatment for the subject, one or more further tests for assessing sepsis risk, a Procalcitonin test, and a Monocyte Distribution Width (MDW) test.

It should be noted that in addition to or instead of a visual or graphical notification 160, also an acoustic or haptic notification 160 may be provided at the user interface 140 or another user interface of the computing device 100.

Generally, by generating the notification 160 informative of the risk for the subject having occult sepsis, HCPs or medical practitioners may be reliably informed about a potential occult sepsis being present or being developed at the subject, optionally within the predetermined period of time. This may enable HCPs to take appropriate actions, for example to initiate an appropriate therapy, to keep the subject under surveillance and/or to perform one or more measurements for one or more health-related parameters. Also, it may be avoided that a subject having only subtle or no symptoms of sepsis is erroneously discharged from an emergency department, clinic or hospital. As a consequence, delays in the treatment can be effectively avoided and a timely treatment and therapy can be ensured.

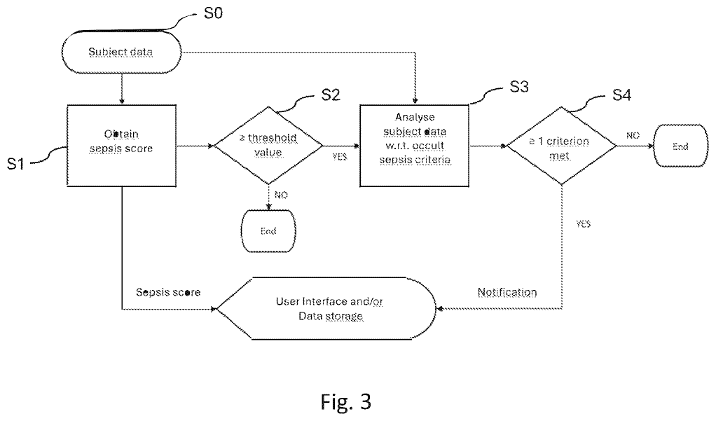

FIG. 3 shows a flow chart illustrating a method of determining, for a subject, a risk of having an occult sepsis according to an exemplary embodiment, for example using the computing device 100 described with reference to FIGS. 1 and 2. Hence, any feature, function or element described with reference to FIGS. 1 and 2 can be a step of the method of FIG. 3, and vice versa.

The exemplary method illustrated in FIG. 3 comprises step S0, in which the set of subject data may be provided to the computing device 100 and/or may be obtained by or received at the computing device 100. This may include, for example, accessing the set of subject data at a memory or data storage 120 of the computing device 100 and/or retrieving the subject data therefrom. Alternatively or additionally, the set of subject data can be retrieved and/or downloaded by the computing device 100 from one or more external data sources 200, such as an electronic health record of the subject.

Step S1 comprises obtaining by, e.g., receiving at, the computing device 100, at least one sepsis score indicative of a risk of the subject developing a sepsis event.

Optionally, obtaining the at least one sepsis score S1 may comprise accessing and/or retrieving the sepsis score e.g. from at least one memory or data storage 120 of the computing device 100, or from the one or more external data sources 200. Retrieving the sepsis score may comprise downloading the sepsis score to the computing device 100. Additionally or alternatively, obtaining the sepsis score may comprise receiving the sepsis score, e.g. from a user or one or more external data sources 200.

Alternatively or additionally, obtaining the at least one sepsis score S1 can comprise determining, computing and/or calculating, based on at least a part of the set of subject data and/or based on supplementary subject data, the at least one sepsis score. In other words, the at least sepsis score may be determined based on or using at least a part of the subject data and/or based on or using supplementary subject data. Therein, the part of the subject data used to determine the sepsis score may at least partly overlap with and/or may at least partly differ from the subject data that is obtained at step S0. Alternatively or additionally, the supplementary subject data used to determine the sepsis score may at least partly overlap with and/or may at least partly differ from the subject data that is obtained at step S0.

In particular, the at least part of the set of subject data and/or the supplementary subject data utilised to determine or compute the sepsis score includes and/or is indicative of one of, a plurality of, or all of: a subject age value, a subject heart rate value, a subject respiratory rate value, a subject diastolic blood pressure value, a subject systolic blood pressure value, a blood pressure value, a subject temperature value, at least one value related to Monocyte Distribution Width (MDW), a White Blood Cell Count (WBC), a subject oxygen saturation value, a lactate value, a partial pressure of CO₂, a neutrophil value, and a Glasgow coma. Other or additional health-related or subject parameters may be used to optionally determine or compute the at least one sepsis score S1, as described above.

Optionally, the set of subject data, at least a part thereof, and/or the supplementary subject data may be obtained by the computing device at step S0 and/or at step S1.

The at least one sepsis score obtained at step S1 may optionally be provided, output and/or displayed at the user interface 140 of the computing device 100, as illustrated in FIG. 2. Alternatively or additionally, the at least one sepsis score may be stored at the data storage 120 and/or at one or more external data sources 200, such as e.g. in an electronic health record.

At step S2, the computing device 100 determines whether the obtained at least one sepsis score meets at least one threshold value for the at least one sepsis score. This may include comparing the sepsis score to the at least one threshold value and coming to a conclusion as to whether the threshold value is reached or exceeded.

As described in more detail hereinabove, the sepsis score may be continuous variable and may be comprised between a minimum and maximum value, wherein the threshold value may for example correspond to at least 40%, at least 50%, at least 60% at least 70% or at least 80% of the maximum value.

Alternatively, the sepsis score may indicate a certain level or tier of risk for sepsis, such as low, medium and high risk, and the threshold value may correspond to or be associated with one or more of these tiers or levels. For instance, the threshold value may indicate at least medium and/or at least high risk for sepsis.

If the threshold value for the sepsis score is not reached or exceeded, it may be concluded that the subject is not at risk for sepsis and also not at risk for occult sepsis.

If the at least one threshold value is reached or exceeded, the method continues at step S4, which includes analysing the set of subject data indicative of a health state of the subject with respect to one or more occult sepsis criteria indicative of a risk of the subject having an occult sepsis.

If none of the occult sepsis criteria or none of one or more subsets of occult sepsis criteria, such as none of subsets 1 to 22 listed in the summary section, are fulfilled, it may be concluded that the subject is not at risk for occult sepsis.

If one or more of the occult sepsis criteria are fulfilled or met by one or more health-related or subject parameters indicated by the set of subject data, a notification 160 informative of the risk for occult sepsis may be generated and/or displayed at the user interface 140, as described in detail hereinabove. Optionally, the information contained in the notification may be stored at a data storage 120 of the computing device and/or one or more external data sources, such as e.g. an electronic health record associated with the subject.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A computer-implemented method of determining, for a subject, a risk of having an occult sepsis, the method comprising:
obtaining at least one sepsis score indicative of a risk of the subject developing a sepsis event;
confirming that the obtained at least one sepsis score meets at least one threshold value for the at least one sepsis score; and
analysing a set of subject data indicative of a health state of the subject with respect to one or more occult sepsis criteria indicative of a risk of the subject having an occult sepsis.

2. The method according to claim 1, wherein the subject data comprises at least one health-related parameter of the subject; and
wherein analysing the subject data with respect to the one or more occult sepsis criteria comprises determining whether the at least one health-related parameter meets at least one of the one or more occult sepsis criteria.

3. The method according to claim 1, wherein the one or more occult sepsis criteria include one or more of:
(a) a temperature equal to or above 36° C.;
(b) a temperature equal to or below 38° C.;
(c) a temperature between 36° C. and 38° C.;
(d) a systolic blood pressure equal to or above 90 mm Hg;
(e) a lactate value equal to or below 2 mmol/L;
(f) a respiratory rate equal to or below 20 breaths/minute;
(g) partial pressure of CO₂ equal to or above 32 mmHg;
(h) a heart rate equal to or below 90 bpm;
(i) a white blood cell count value equal to or above 4×10⁹/L;
(j) a white blood cell count value equal to or below 12×10⁹/L;
(k) a white blood cell count value between 4×10⁹/L and 12×10⁹/L;
(l) neutrophil bands equal to or below 10%, or equal to or below 10% immature forms of neutrophils;
(m) one or more complaints selected from the group consisting of fever, chills, rigors, cough, soft tissue, and infection;
(n) exclusion of Systemic Inflammatory Response Syndrome;
(o) a systolic blood pressure equal to or above 100 mmHg;
(p) a respiratory rate equal to or below 21 breaths/minute;
(q) a Glasgow coma score equal to or above 14 and/or a Glasgow coma score equal to 15.

4. The method according to claim 1, wherein analysing the set of subject data with respect to the one or more occult sepsis criteria comprises:
determining that at least occult sepsis criteria (a)-(c), at least occult sepsis criteria (a)-(d), at least occult sepsis criteria (a)-(d) and (f), at least occult sepsis criteria (a)-(d) and (f) and (k), at least occult sepsis criteria (a)-(d) and (f) and (k)-(l), at least occult sepsis criteria (a)-(d) and (f) and (k)-(m), at least occult sepsis criteria (a)-(f)), at least occult sepsis criteria (a)-(e), at least occult sepsis criteria (a)-(f), at least occult sepsis criteria (a)-(f) and (k), at least occult sepsis criteria (a)-(f) and (k)-(l), at least occult sepsis criteria (a)-(f) and (k)-(m), at least occult sepsis criteria (a)-(g), at least occult sepsis criteria (a)-(h), at least occult sepsis criteria (a)-(i), at least occult sepsis criteria (a)-(j), at least occult sepsis criteria (a)-(k), at least occult sepsis criteria (a)-(l), at least occult sepsis criteria (a)-(m), at least occult sepsis criteria (a)-(n), at least occult sepsis criteria (a)-(o), at least occult sepsis criteria (a)-(p), or occult sepsis criteria (a)-(q) are each fulfilled.

5. The method according to claim 3, wherein analysing the set of subject data with respect to the one or more occult sepsis criteria comprises determining that at least occult sepsis (a)-(f) and (k)-(m) are each fulfilled.

6. The method according to claim 1, wherein analysing the set of subject data with respect to the one or more occult sepsis criteria comprises predicting if the subject is having occult sepsis or not.

7. The method according to claim 1, further comprising:
generating, upon determining that at least a part of the subject data and/or at least one health-related parameter comprised in the subject data meets at least one of the one or more occult sepsis criteria, a notification informative of the risk for the subject having occult sepsis.

8. The method according to claim 1, wherein the notification is notifiable to a medical practitioner via a user interface of a computing device.

9. The method according to claim 7, wherein the notification includes one or more of a text-based notification, a sound-based notification, a haptic notification, and a visual notification.

10. The method according to claim 7, wherein the notification includes a recommendation or instruction to order one or more of a lactate test, antibiotic treatment for the subject, one or more further tests for assessing sepsis risk, a Procalcitonin test, and a Monocyte Distribution Width (MDW) test.

11. The method according to claim 1, wherein confirming that the obtained at least one sepsis score meets the at least one threshold value for the at least one sepsis score comprises:
comparing the obtained at least one sepsis score to the at least one threshold value for the sepsis score; and
determining that the obtained at least one sepsis score reaches, exceeds or falls below the at least one threshold value for the sepsis score.

12. The method according to claim 1, wherein the at least one sepsis score is a numerical measure indicative of the risk for the subject developing the sepsis event, optionally within a predetermined period of time.

13. The method according to claim 1, wherein the sepsis score ranges from a minimum sepsis score value to a maximum sepsis score value, and
wherein the at least one threshold value for the sepsis score is at least 40% of the maximum sepsis score value, at least 50% of the maximum sepsis score value, or at least 60% of the maximum sepsis score value, or at least 70% of the maximum sepsis score value, or at least 80% of the maximum sepsis score value, or at least 90% of the maximum sepsis score value.

14. The method according to claim 1, further comprising:
determining, based on at least a part of the set of subject data and/or supplementary subject data, the at least one sepsis score.

15. The method according to claim 14, wherein the at least one sepsis score is determined based on at least one machine learning model.

16. The method according to claim 14, wherein determining the at least one sepsis score includes computing the risk for the subject developing the sepsis event based on at least one machine learning model, wherein the at least one machine learning model is configured to receive and process input data, the input data comprising and/or being based on at least a part of the set of subject data and/or based on supplementary subject data.

17. The method according to claim 14, wherein the at least part of the set of subject data and/or the supplementary subject data includes and/or is indicative of at least one of: a subject age value, a subject heart rate value, a subject respiratory rate value, a subject diastolic blood pressure value, a subject systolic blood pressure value, a blood pressure value, a subject temperature value, at least one value related to Monocyte Distribution Width (MDW), a White Blood Cell Count (WBC) value, a subject oxygen saturation value, a lactate value, a partial pressure of CO₂ value, a neutrophil value, and a Glasgow coma score value.

18. A computing device including one or more processors for data processing, wherein the computing device is configured to carry out steps of the method according to claim 1.

19. A computer program comprising instructions, which, when the program is executed by a computing device, cause the computing device to carry out the steps of the method according to claim 1.

20. A non-transitory computer-readable medium storing the computer program according to claim 19.
